(19) United States
(12) Patent Application Publication
(10) Pub. No.: US 2025/0312365 A1
(43) Pub. Date: Oct. 9, 2025

(54) METHODS FOR INHIBITION OF SQUAMOUS CARCINOMA CELLS USING CERAMIDE MODULATORS

(71) Applicant: The Trustees of Indiana University, Bloomington, IN (US)

(72) Inventors: Alexandru Movila, Fishers, IN (US); Chiaki Yamada, Bloomington, IN (US); Frank Nichols, Farmington, CT (US)

(21) Appl. No.: 19/173,983

(22) Filed: Apr. 9, 2025

Related U.S. Application Data
(60) Provisional application No. 63/631,934, filed on Apr. 9, 2024.

(51) Int. Cl. A61K 31/688; A61K 35/74; A61P 35/00

(52) U.S. Cl. CPC A61K 31/688; A61K 35/74; A61P 35/00

(57) ABSTRACT

Disclosed herein are methods and compositions useful for selectively inhibiting the viability, growth, or metastasis of oral cancer cells, including oral squamous cell carcinomas. Disclosed methods involve contacting such cells with an effective amount of a ceramidase modulator. The ceramidase modulators include at least one phosphorylated dihydroceramide and phospholipid compounds, which can inhibit the viability of the oral cancer cells, for example, by increasing intracellular accumulation of a ceramide and/or a dihydroceramide in the cells or inhibiting the gene expression of ceramidases. Also disclosed are pharmaceutical compositions including ceramidase modulators, such as compositions including phosphorylated dihydroceramide and phospholipid compounds isolated from oral and gut bacteria species, respectively.

### CROSS-REFERENCE TO RELATED APPLICATIONS

This application claims the benefit under 35 U.S.C. §199(e) of U.S. Provisional Application No. 63/631,934 entitled METHODS FOR INHIBITION OF SQUAMOUS CARCINOMA CELLS USING CERAMIDE MODULATORS, filed on Apr. 9, 2024 which is incorporated by reference in its entirety.

### STATEMENT OF GOVERNMENTAL RIGHTS

This invention was made with government support under AG068595 and AG064003 awarded by National Institutes of Health. The Government has certain rights in the invention.

### FIELD OF THE INVENTION

This disclosure generally relates to inhibition and treatment of oral cancer cells, such as oral squamous cell carcinoma, tongue squamous cell carcinoma, and head and neck squamous cell carcinoma. More specifically, the disclosure relates to use of phosphorylated dihydroceramides to reduce the proliferation, growth, and metastasis of oral cancer cells.

### BACKGROUND

Mouth cancers most commonly begin in the flat, thin cells (squamous cells) that line the lips and the inside of the mouth. More than 90 percent of mouth cancers are squamous cell carcinoma. Current treatment options for oral squamous cell carcinoma include chemotherapy, immunotherapy, or advanced radiation techniques, including proton or intensity-modulated radiation therapy. However, each of these current treatment options have the disadvantage of impacting healthy (non-cancerous) cells in the treatment area. There is need for targeted treatment of squamous cell carcinoma. Aspects of the invention disclosed herein address these needs.

### INCORPORATION BY REFERENCE

Each patent, publication, and non-patent literature cited in the application is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art.

### SUMMARY OF THE INVENTION

A first aspect of the invention includes methods for inhibiting any of the growth, viability, migration, and metastasis of oral cancer cells.

A second aspect of the invention includes compositions containing a phosphorylated dihydroceramide.

A first embodiment is a method for inhibiting viability of oral cancer cells including contacting the oral cancer cells with an effective amount of a ceramidase modulator, thereby inhibiting the viability of the oral cancer cells, where the ceramidase modulator includes a phosphorylated dihydroceramide.

A second embodiment is a method where inhibiting the viability of oral cancer cells is verified by selectively inhibiting a viability marker in a sample of the oral cancer cells, as determined by comparing a same viability marker in a sample of normal oral epithelial cells contacted by the effective amount of the ceramidase modulator.

A third embodiment is a method where the viability marker is selected from cell adhesion, growth, survival, viability, proliferation, migration, invasion, metastasis, or a combination thereof.

A fourth embodiment is a method where the method includes inhibiting the proliferation of the oral cancer cells.

A fifth embodiment is a method, where the method includes inhibiting the migration of the oral cancer cells.

A sixth embodiment is a method where the method includes inhibiting the gene expression of NF-kB, MMP-2, IL6, or a combination thereof, in the oral cancer cells.

A seventh embodiment is a method where the method includes increasing the concentration of a ceramide and/or a dihydroceramide in the oral cancer cells.

An eighth embodiment is a method, where the ceramide is selected from the group consisting of C20:0, C26:0, and C26:1; and the dihydroceramide is any one or more of dhC14:0, dhC16:0, dhC18:0, dhC20:0, dhC20:1, dhC22:1, dhC22:0, dhC24:1, dhC24:0, and dhC26:1.

A ninth embodiment is a method where the method includes inhibiting the gene expression of a ceramidase in the oral cancer cells.

A tenth embodiment is a method where the ceramidase is selected from ASAH1 and ACER2.

An eleventh embodiment is a method where the phosphorylated dihydroceramide includes phosphoethanolamine dihydroceramide (PEDHC), phosphoglycerol dihydroceramide (PGDHC), a 15:0-15:0PE phospholipid, or an analog thereof.

A twelfth embodiment is a method where the oral cancer cells comprise oral squamous cell carcinoma, tongue squamous cell carcinoma, head and neck squamous carcinoma, or a combination thereof.

A thirteenth embodiment is a method where the oral cancer cells or the sample of the oral cancer cells is derived from a subject having cancer.

A fourteenth embodiment is a method where the subject has mouth cancer, lip cancer, tongue cancer, head and neck cancer, or a combination thereof.

A fifteenth embodiment is a method where the subject is a mammal, and where the mammal is a human.

A sixteenth embodiment is a pharmaceutical composition including a ceramidase modulator and a pharmaceutically acceptable excipient, where the ceramidase modulator includes a phosphorylated dihydroceramide.

A seventeenth embodiment is a method where the phosphorylated dihydroceramide is purified from a bacterial cell.

An eighteenth embodiment is a method where the phosphorylated dihydroceramide is purified from Porphyromonas gingivalis.

A nineteenth embodiment is a method where the ceramidase modulator includes phosphoethanolamine dihydroceramide (PEDHC) or phosphoglycerol dihydroceramide (PGDHC).

A twentieth embodiment is a method where the composition includes phosphoethanolamine dihydroceramide (PEDHC), phosphoglycerol dihydroceramide (PGDHC), an analog thereof, or a combination thereof.

A twenty-first embodiment is a method where the phospholipid is purified from Akkermansia muciniphila.

A twenty-second embodiment is a method where the ceramidase modulator includes a 15:0-15:0PE phospholipid.

A twenty-third embodiment is a method where the composition includes a 15:0-15:0PE phospholipid or an analogue thereof.

A twenty-fourth embodiment is a method where the composition includes phosphoethanolamine dihydroceramide (PEDHC), phosphoglycerol dihydroceramide (PGDHC), a 15:0-15:0PE phospholipid, an analog thereof, or a combination thereof.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A-1C show the chemical structure of exemplary dihydroceramide lipids. FIG. 1A illustrates an exemplary P. gingivalis phosphoglycerol dihydroceramide (PGDHC) lipids, with the asterisk indicating that the 18 carbon long chain is a straight aliphatic chain rather than isobranched; FIG. 1B illustrates an exemplary P. gingivalis phosphoethanolamine dihydroceramide (PEDHC) lipid; FIG. 1C illustrates an exemplary gut bacterial phospholipid Akkermansia muciniphila 1-(12S-methylmyristoyl)-2-(13-methylmyristoyl)-sn-glycero-3-phosphoethanolamine (a15:0-15:0PE).

FIG. 2 shows bar graphs representing the effect of PGDHC (purified from Porphyromonas gingivalis), on the cell viability of OBA9 epithelial cells, oral squamous OECM1 cells, SCC4 cells, and SCC9 cells, as detected by an in vitro WST-1 assay (*p<0.05, **p<0.01).

FIG. 3A shows bar graphs representing the effect of PGDHC (purified from Porphyromonas gingivalis), on the expression of OECM1 genes associated with degradation of oral squamous cell basement membrane and extracellular matrix, including NFkB, MMP2, ADAM17, and IL6 (*p<0.05, **p<0.01).

FIG. 3B shows images of control OECM1 cells and the effects of PGDHC (0.1 μg/ml, and 1 μg/ml) on OECM1 cell migration in a wound healing scratch test.

FIG. 3C is a bar graph representing the effect of PGDHC on OECM1 cell migration in a wound healing scratch test (*p<0.05).

FIG. 3D shows D&E staining of control OECM1 cells and the effects of PGDHC (1 μg/ml) on OECM1 cell migration in an insert transmigration assay.

FIG. 3E is a bar graph representing the effect of PGDHC on the number of migrated OECM1 cells in an insert transmigration assay (*p<0.05).

FIG. 4 shows bar graphs representing the in vitro effects of PGDHC on the expression of SCC4 cells genes implicated in basement membrane and extracellular matrix degradation of oral squamous cells, including NFkB, MMP2, ADAM17, and IL6.

FIG. 5A shows bar graphs representing the in vitro effects of spingosine-1-phosphate (S1P) alone or in combination with PGDHC (purified from Porphyromonas gingivalis) on the expression of OECM1 genes implicated in basement membrane and extracellular matrix degradation of oral squamous cells, including NFkB, MMP2, ADAM17, and IL6 (*p<0.05, **p<0.01, ***p<0.001).

FIG. 5B shows images of OECM1 cells exposed to spingosine-1-phosphate (S1P) alone or in combination with PGDHC in a wound healing migration assay.

FIG. 5C is a bar graph representing percent OECM1 cell migration in a wound healing migration assay following exposure to spingosine-1-phosphate (S1P) alone or in combination with PGDHC relative to a control (**p<0.01, ***p<0.001).

FIG. 6A shows Western blot analysis of p-STAT3, STAT3, and GADPH in a time course study on the effects of PGDHC in OECM1 cells.

FIG. 6B shows Western blot analysis of E-cadherin, N-cadherin, Vimentin, and GADPH on the effects of PGDHC in OECM1 cells for 24 hours.

FIG. 7A is a bar graph showing percent cell viability of OECM1 cells exposed to 0.1 μg/ml, 1 μg/ml, 2 μg/ml, and 5 μg/ml ceramide C6 normalized to a control sample (no C6 exposure).

FIG. 7B shows a panel of bar graphs representing the effects of C6 exposure on the gene expression of ceramidases ASAH1 (left) ACER2 (center) ACER3 (right) in OECM1 cells relative to a control sample.

FIG. 8 has three panels of bar graphs showing the effect of PGDHC, purified from Porphyromonas gingivalis, on the gene expression of ASAH1 (left), ACER2 (middle), and ACER3 (right) in OECM1 cells.

FIG. 9 has three panels of bar graphs showing the effect of PGDHC, purified from Porphyromonas gingivalis, on the gene expression of ASAH1 (left), ACER2 (middle), and ACER3 (right) in SCC4 cells.

FIG. 10A is a bar graph showing the level of ceramide and dihydroceramide species in OECM1 cells following exposure to control conditions and PGDHC (0.1 μg/ml and 1 μg/ml).

FIG. 10B is a bar graph showing the level of dihydroceramide species in OECM1 cells following exposure to control conditions and PGDHC (0.1 μg/ml and 1 μg/ml) (*p<0.05 and **p<0.01).

FIG. 10C shows bar graphs representing the comparative change in ceramide and dihydroceramide levels in OECM1 cells following exposure to control conditions and PGDHC (0.1 μg/ml and 1 μg/ml) (**p<0.01 and ***p<0.001).

FIG. 11A is a plot showing the correlation between the levels of ASAH1 mRNA and the fold change of ceramide in OECM1 cells.

FIG. 11B is a plot showing the correlation between the levels of ASAH1 mRNA and the fold change of dihydroceramide in OECM1 cells.

FIG. 11C is a plot showing the correlation between the levels of ACER2 mRNA and the fold change of ceramide in OECM1 cells.

FIG. 11D is a plot showing the correlation between the levels of ACER2 mRNA and the fold change of dihydroceramide in OECM1 cells.

FIG. 12A is a panel of bar graphs representing the percent cell viability of OECM1 cells exposed to 0.1 μg/ml, 1 μg/ml, 2 μg/ml, and 5 μg/ml C16 and dh-C16.

FIG. 12B shows bar graphs representing gene expression of ceramidases ASAH1, ACER2, and ACER3 in OECM1 cells exposed to 1 μg/ml exposed to C16 and dh-C16 relative to a sham control.

FIG. 13A is a table setting out demographic characteristics and the OSCC grade of the sample obtained for histological evaluation.

FIG. 13B are representative images of the hematoxylin & eosin (H&E) staining of the tongue with OSCC lesions and matched adjacent normal control tissue obtained from the same patient.

FIG. 13C are representative images of immunohistochemical (IHC) staining for acid ceramidase and S1P of OSCC and matched adjacent normal control tissue collected from the same patient.

FIG. 13D shows bar graphs representing quantification of the H-Score for acid ceramidase and S1P in human OSCC, and matched adjacent normal control tissue. Scale bar: 200 μm. **p<0.01, detected by two-way ANOVA with Tukey's post hoc.

FIG. 13E shows the Pearson correlation coefficient scatter plot between H-Score of acid ceramidase and S1P in human OSCC, and matched adjacent normal control tissue from the same patients.

FIG. 14A shows a bar graph of ASAH1 mRNA expression in different OSCC cell lines oral cavity (OECM1) and tongue (SCC9 and SCC4), and in healthy epithelial OBA9 cells (OBA9). Scale bar—50 μm. N=3, *p<0.05, **p<0.01, ***P<0.001 obtained by ANOVA with Tukey's post hoc analysis.

FIG. 14B are representative images of acid ceramidase levels detected in different OSCC and healthy epithelial cells by confocal microscopy (anti-acid ceramidase (aCDase) mAb; Abcam: dilution 1:1000), actin (phalloidin), and nuclei (Draq5) are shown in green, blue, and red, respectively.

FIG. 14C shows a bar graph of levels of acid ceramidase in different OSCC and healthy OBA9 cells quantified as corrected total cell fluorescence based on the confocal image FIG. 14B. Scale bar—50 μm. N=3, *p<0.05, **p<0.01, ***P<0.001 obtained by ANOVA with Tukey's post hoc analysis.

FIG. 15A shows a bar graph of cell viability in the presence of LCL521 at concentrations 0.1, 1.0, and 10 μM in different OSCC cell lines oral cavity (OECM1) and tongue (SCC9 and SCC4), and in healthy epithelial OBA9 cells (OBA9). N=4 samples/condition. *p<0.05, **p<0.01 by ANOVA with Tukey's post hoc test. NS-non-significant.

FIG. 15B shows a bar graph of cell viability in the presence of a 15:0-i15:0 PE at concentrations of 0.1, 1.0 and 2.0 μM in different OSCC cell lines oral cavity (OECM1) and tongue (SCC9 and SCC4), and in healthy epithelial OBA9 cells (OBA9). N=4 samples/condition. *p<0.05, **p<0.01 by ANOVA with Tukey's post hoc test. NS—non-significant.

FIG. 16A and FIG. 16B shows plot displays of the enriched metabolic pathways in OECM1 (FIG. 16A) and SCC9 (FIG. 16B) cells exposed to PGDHC (1 μg/ml) for 6 h in vitro, with their corresponding significance levels represented on the x-axis. The significance threshold (p<0.05) is denoted by the dashed line. The size of the dots reflects the number of metabolites that showed statistical differences between the sample groups relative to the Control group (N=5/condition). Additionally, the color of the dots represents the enrichment, which indicates the extent to which the metabolites in the pathway were clustered amongst the compounds with the most positive fold-changes (positive enrichments), the most negative fold-changes (negative enrichments), or uniformly distributed across high and low fold-change compounds (enrichment values near 0.0).

FIG. 16C heat map based on the data presented in FIGS. 16A and 16B showing PGDHC isolated from Porphyromonas gingivalis inhibits folate metabolism in human OECM1 and SCC9 oral cancer cells, respectively, in vitro.

FIG. 16D heat map based on the data presented in FIGS. 16A and 16B showing PGDHC isolated from Porphyromonas gingivalis inhibits arginine and proline metabolism in human OECM1 and SCC9 oral cancer cells, respectively, in vitro.

FIG. 16E heat map based on the data presented in FIGS. 16A and 16B showing PGDHC isolated from Porphyromonas gingivalis inhibits glycine and serine metabolism in human OECM1 and SCC9 oral cancer cells, respectively, in vitro.

FIG. 16F heat map based on the data presented in FIGS. 16A and 16B showing PGDHC isolated from Porphyromonas gingivalis inhibits de novo Triacylglycerol biosynthesis metabolism in human OECM1 and SCC9 oral cancer cells, respectively, in vitro.

FIG. 16G heat map based on the data presented in FIGS. 16A and 16B showing PGDHC isolated from Porphyromonas gingivalis inhibits mitochondrial electron transport metabolism in human OECM1 and SCC9 oral cancer cells, respectively, in vitro.

FIG. 17A are heat maps demonstrating that PGDHC altered the Warburg effect in OSCM-1 and SCC9 oral squamous cancer cells. Cells were stimulated with PGDHC (1 μg/ml) for 6 h and the untargeted metabolomic analysis was performed using LC/MS mass-spectrometry. Polar and lipid metabolites were analyzed with HILIC/MS and LC/MS, respectively. Mass spectrometry analysis was completed with a mass range of 67-1500 Da with 1 scan/sec and a mass resolution of 120,000 in both positive and negative ionization modes. MS/MS data were acquired in a data-dependent iterative fashion with a 1.3 m/z isolation window.

FIG. 17B is a heat map demonstrating no effect of PGDHC on the Warburg effect in healthy OBA9 cells. Cells were stimulated with PGDHC (1 μg/ml) for 6 h and the untargeted metabolomic analysis was performed using LC/MS mass-spectrometry. Polar and lipid metabolites were analyzed with HILIC/MS and LC/MS, respectively. Mass spectrometry analysis was performed with a mass range of 67-1500 Da, at a scan rate of 1 scan per second, and a mass resolution of 120,000 in both positive and negative ionization modes. MS/MS data were acquired in a data-dependent iterative fashion with a 1.3 m/z isolation window.

FIGS. 18A and 18B are graphs depicting altered levels of adenosine triphosphate (ATP) and glucose in OECM1, SCC9, and OBA9 cells. N=5, *p<0.05

FIG. 19A is a bar graph depicting expression patterns of ASAH1 mRNA in OECM1and SCC9 cells exposed to different concentrations of a15:0-15:0PE. Scale bar-50 μm. N=3, *p<0.05, **p<0.01, ***P<0.001 obtained by ANOVA with Tukey's post hoc analysis

FIG. 19B are representative confocal microscopy images of SCC9 stained for acid ceramidase (green), actin (blue), nuclei (red) using anti-acid ceramidase Ab, phalloidin and Draq5, respectively.

FIG. 19C is a bar graph of levels of acid ceramidase in SCC9 cells quantified as corrected total cell fluorescence based on the confocal images (see FIG. 19B). Scale bar—50 μm. N=3, *p<0.05, **p<0.01, ***P<0.001 obtained by ANOVA with Tukey's post hoc analysis.

FIG. 20A are plot displays of the enriched pathways identified in the analysis of human OSCC tongue SCC9 cells exposed to the a15:0-15:0PE phospholipid, isolated from the gut Akkermansia muciniphila bacteria, with their corresponding significance levels represented on the x-axis. The significance threshold (p<0.05) is denoted by the grey dashed line. The size of the dots reflects the number of metabolites that showed statistical differences between the sample groups relative to the Control group (N=5/condition). Additionally, the color of the dots represents the enrichment, which indicates the extent to which the metabolites in the pathway were clustered amongst the compounds with the most positive fold-changes (positive enrichments), the most negative fold-changes (negative enrichments), or uniformly distributed across high and low fold-change compounds (enrichment values near 0.0).

FIG. 20B is a heat map created based on the data presented in FIG. 20A showing the a15:0-15:0PE phospholipid, isolated from the gut Akkermansia muciniphila bacteria, inhibits amino fatty acids metabolism in human SCC9 oral cancer cells in vitro.

FIG. 20C is a heat map created based on the data presented in FIG. 20A showing the a15:0-15:0PE phospholipid, isolated from the gut Akkermansia muciniphila bacteria, inhibits arginine and proline amino acids metabolism in human SCC9 oral cancer cells in vitro.

FIG. 20D is a heat map created based on the data presented in FIG. 20A showing the a15:0-15:0PE phospholipid, isolated from the gut Akkermansia muciniphila bacteria, inhibits Vitamin B6 metabolism in human SCC9 oral cancer cells in vitro.

### DETAILED DESCRIPTION

Squamous cell carcinoma is the most common type of oral cancer, and oral squamous cell carcinoma (OSCC) is one of the most common malignant cancers of the head and neck. OSCC may develop on the mucosal epithelium of the oral cavity, including epithelial tissue that lines the mouth, tongue, gums, and lips. OSCC can also develop at other mucous membranes of the oral cavity, including the buccal mucosa, palate, mouth floor, jawbone, and salivary gland. Depending on the severity of the cancer, an OSCC patient can experience changes in appearance and impaired pronunciation, swallowing, and flavor perception. See, e.g., Bugshan et al., Version 1. F1000Res. 2020; 9: 229 and Tan et al., Int J Oral Sci. 2023 Sep. 22;15(1):44.

Notably, OSCC can alter cell sphingolipid metabolism, such as by increasing species that lead to proliferation, such as sphingosine-1-phosphate (S1P), while also decreasing antiproliferative species, such as ceramide. The ceramide/S1P ratio is regulated by acid ceramidase (ASAH1). See, e.g., Doan et al., Oncotarget. 2017;8(68):112662-112674 and Maceyka et al., Trends Cell Biol. 2012;22(1):50-60. Diminishing ceramidase gene expression, such as that of ASAH1, can lead to the accumulation of intracellular ceramide in OSCC. Accordingly, this approach has emerged as a potential objective for oral cancer therapy.

Previously, oral periodontal bacteria Porphyromonas ginigvalis was shown to inhibit expression patterns of ASAH1 in epithelial cells in vitro (Azuma et al., Biochem Biophys Res Commun. 2018 Jan. 22;495(4):2383-2389). Porphyromonas ginigvalis is a unique bacterial species which produces dihydroceramide sphingolipids, including phosphoethanol dihydroceramide (PEDHC) and phosphoglycerol dihydroceramide (PGDHC) (Nichols et al., PLoS ONE 2011;6(2): e16771).

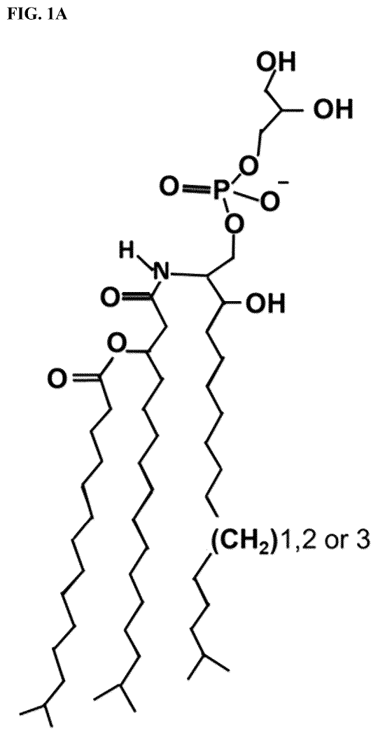

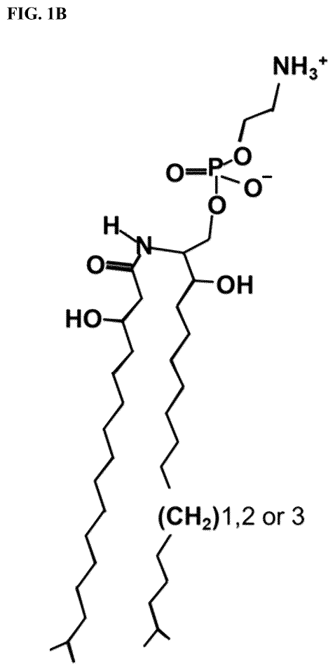

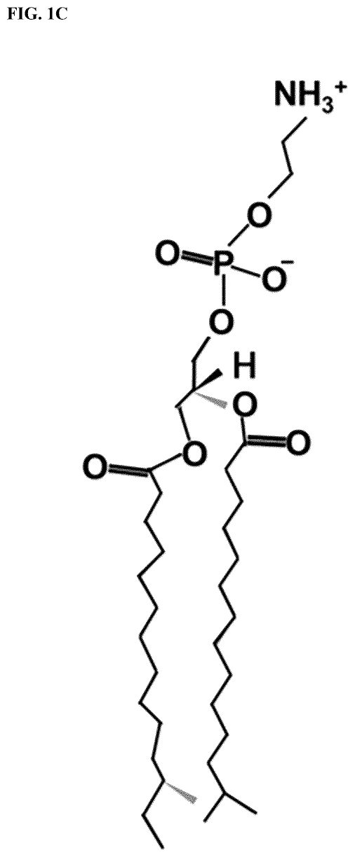

FIGS. 1A, and 1B show the chemical structure of P. gingivalis phosphoglycerol dihydroceramide and phosphatidyl-ethanolamine lipid classes. Importantly, multiple gut and oral bacterial species produce PEDHC. However, PGDHC is primary produced by Porphyromonas ginigvalis. Additionally, it was demonstrated that phosphoethanol dihydroceramide produced by bacteria can elevate accumulation of ceramide species in host cells (Johnson et al., Nature Communications 2020;11, 2471). FIG. 1C shows the chemical structure of phospholipid from the gut bacteria Akkermansia muciniphila, the phospholipid referred to herein as a15:0-15:0PE.

Applicant performed in vitro studies to determine the effect of PEDHC and PGDHC on the proliferation of oral cancer cells via downregulation of acid ceramidase, thereby contributing to the accumulation of intracellular ceramides. PEDHC inhibited the proliferation of OECM1 cells, a human oral squamous cell carcinoma cell line, via downregulation of ASAH1 expression (Yamada et al., J Cell Mol Med. 2023 May;27(9):1290-1295). Surprisingly, preliminary findings showed that while PGDHC dramatically inhibited ASAH1 expression and led to the accumulation of dihydroceramide in OECM1 cells, these same effects were not observed in healthy oral epithelial cells. Furthermore, PGDHC diminished metabolites in at least five different cell survival metabolomic pathways in OSCC cells but not in healthy cells. The metabolomic pathways included folate metabolism, arginine and proline metabolism, triacylglycerol biosynthesis de novo, mitochondrial election transport chain, and glycine and serine metabolism. The a15:0-15:0PE similarly exhibited a dramatic inhibition of ASAH1 expression and diminished metabolites in at least three different metabolomic pathways in OSCC, including amino fatty acid, vitamin B6 metabolism, and arginine and proline metabolism. In contrast to current approaches for undermining the viability of oral squamous cell carcinoma and treating OSCC, the present disclosure relates in some aspects to methods and compositions for selectively interfering with any of the growth, viability, proliferation, migration, and metastasis of oral squamous cell carcinoma.

### Methods

In some aspects, provided herein are methods for inhibiting viability and proliferation of an oral cancer cell, such as by contacting the oral cancer cell with an effective amount of a ceramidase modulator. In some examples, inhibiting the oral cancer cell involves inhibiting any of oral cancer cell adhesion, growth, survival, viability, proliferation, migration, and metastasis. In some embodiments, the oral cancer cell is an oral squamous cell carcinoma, tongue squamous cell carcinoma, gingival squamous cell carcinoma, or a head and neck cell carcinoma. In some embodiments, inhibiting the oral cancer cell can be verified by assessing a viability marker, the viability marker can be a measure for any of adhesion, growth, survival, viability, proliferation, migration, and metastasis.

In some embodiments, the ceramide modulator includes a biologically active lipid. In some embodiments, the biologically active lipid is a sphingolipid, such as a phosphorylated dihydroceramide, e.g., phosphoethanolamine dihydroceramide (PEDHC), phosphoglycerol dihydroceramide (PGDHC), or phospholipid a15:0-15:0PE. In some examples, the biologically active lipid can be isolated, purified, or derived from a bacterial cell. Preferably, the biologically active lipids are isolated, purified, or derived from Porphyromonas gingivalis or Akkermansia muciniphila. In other examples, the biologically active lipid can be synthesized or generated under laboratory or industrial conditions.

In some examples, verifying the inhibition of oral cancer cells involves assessing a viability marker in a sample of oral cancer cells, such as a sample including oral squamous cell carcinoma. The viability marker in the sample of oral cancer cells can be determined according to various control samples. For example, inhibition of the viability marker in a sample of oral cancer cells contacted by the ceramidase modulator can be determined by comparing levels of the same viability marker in an untreated control sample of oral cancer cells. In another example, inhibition of a viability marker in a sample of oral cancer cells contacted by the ceramidase modulator can be determined by assessing the viability marker prior to the contacting step and at one or more time points after the contacting step, i.e., by assessing the same sample of cells. Additionally, inhibition of a viability marker in a sample of oral cancer cells can be determined by comparing the same viability marker in a sample of normal or healthy oral mucosal cells that have been contacted with the same or similar amount of the ceramidase modulator under comparable conditions.

In some embodiments, the control sample is derived from normal oral tissue, for example from a healthy tissue sample from the same patient as the cancerous sample, or from subject or subjects without OSCC. In some embodiments, the oral tissue sample comprises buccal mucosa or cheek, floor of the mouth (FOM), tongue, alveolar, palate, gingival or retromolar tissue. One of skills in the art would recognize methods for distinguishing normal, healthy oral mucosal cells from oral cancer cells, such as squamous cell carcinomas. For example, methods for detecting oral cancer and oral cancer biomarkers are described, e.g., by US 2023/0203493 A1, US 2009/0202624 A1, WO 2018/031545 A1, WO 2023/017543 A1, US 2013/0303826 A1, WO 2008/051374 A2, WO 1998/025615 A1.

In preferred embodiments, disclosed methods involve selectively inhibiting a viability marker of an oral cancer cell, such as a squamous cell carcinoma, where selective inhibition is determined by comparing the viability marker in a sample of oral cancer cells contacted by the ceramidase modulator to a sample of healthy oral tissue also contacted by the ceramidase modulator. In some examples, selective inhibition is demonstrated by observing inhibition of a viability marker in a sample of oral cancer cells contacted by the ceramidase modulator and determining that applying the same treatment has no detectable effect on the same viability marker in a sample of healthy oral tissue.

In preferred embodiments, methods involve significantly altering and/or diminishing the presence of metabolites of metabolomic pathways critical to cancer cell survival. In some embodiments, method involve diminishing one or more of the following metabolomic pathways: folate metabolism, arginine and proline metabolism, glycine and serine metabolism, de novo triacylglycerol biosynthesis, mitochondrial election transport chain, amino fatty acid, or vitamin B6 metabolism.

Measuring markers of growth, viability, proliferation, migration, and metastasis can be determined according to methods available to one of skill in the art. For example, Yamada et al., J Cell Mol Med. 2023 May;27(9):1290-1295, Wang et al., BMC Cancer. 2023 Jul. 17;23(1):668 and Zheng et al., Bioengineered. 2024 December; 15 (1): 2299555 all present various techniques for evaluating the proliferation, progression, invasion, and tumorigenesis of oral squamous cell carcinoma cells.

In specific examples, colorimetric assays can be used to assess cellular viability and proliferation of cell cultures, such as MTT, WST-1, and resazurin assays. See, e.g., Präbst et al., Methods Mol Biol. 2017:1601:1-17. As presented in Example 1, a WST-1 assay unexpectedly revealed the selective inhibitory activity of PGDHC on oral carcinoma cells. In comparison, the tested levels of PGDHC did not have a detectable effect on the proliferation of healthy human gingival epithelial control cells.

In vitro assays can be used to evaluate cell migration, including scratch-wound assays and wound healing assays, as in Xu et al., Exp Ther Med. 2023 October; 26(4):460. See also Liang et al., Nat Protoc. 2007;2(2):329-33 and Martinetti & Ronzato, Methods Mol Biol. 2020:2109:225-229. Additionally, the genetic components of cell migration and invasion can be evaluated. For example, MMP-2, ADAM-17 and IL-6 are associated with degradation of the basement membranes and extracellular matrix in OECM-1 cells. See, e.g., Yamada et al., J Cell Mol Med. 2023 May; 27(9):1290-1295.

In some embodiments, disclosed methods involve modulating the cellular levels of ceramides, including dihydroceramides, and/or modulating the cellular levels of ceramidases. Ceramides are important bioactive lipids which influence several biological processes, ranging from cell proliferation to apoptosis. See, e.g., Wu et al., Neuromol Med 1200 2010;12:320-30, Vitner et al., Handb Exp Pharmacol 2013:405-19, and Horres & Hannun, Neurochem Res 2012;37:1137-49. The structure and function of ceramides is described, e.g., by Castro et al., Prog Lipid Res. 2014 April:54:53-67.

Ceramidases regulate the activity of sphingolipids and ceramides by degrading the lipid molecules. For example, ceramidase enzymes can cleave fatty acid from ceramide and produce sphingosine, thereby controlling the interconversion of the two lipids. Five human ceramidases encoded by five different genes have been identified, including acid ceramidase (AC), neutral ceramidase (NC), alkaline ceramidase 1 (ACER1), alkaline ceramidase 2 (ACER2), and alkaline ceramidase 3 (ACER3). Ceramidases are classified according to their optimal pH for catalytic activity. Ceramidases, such as ASAH1, ASAH2, ASAH2B, ASAH2C, ACER1, ACER2, and ACER3, are reviewed by Coant et al., Adv Biol Regul. 2017 January; 63: 122-131.

In some embodiments, disclosed methods, such as contacting an oral squamous cell carcinoma with a ceramidase modulator, promote the cellular accumulation of ceramides in the oral squamous cell carcinoma. In some examples, disclosed methods promote the accumulation, such as increase the cellular levels, of antiproliferative ceramides in oral squamous cell carcinoma. The cellular concentration of any of the following ceramides can be measured, including C12:0, C14:0, C16:0, C18:0, C18:1, C20:0, C22:0, C22:1, C24:0, C24:1, C26:0 and C26:1, among others. Additionally, the cellular concentration of any of the following dihydroceramides (dh) can be measured, including dhC12:0, dhC14:0, dhC16:0, dhC18:0, dhC18:1, dhC20:0, dhC22:0, dhC22:1, dhC24:0, dhC24:1, dhC26:0 and dhC26:1 among others.

Cellular levels of ceramides and dihydroceramides, and cellular accumulation or increase thereof, can be determined according to known methods, such as mass spectrometry. For example, Scherer et al., J Lipid Res. 2010 July; 51(7): 2001-2011 and Smeden et al., J Lipid Res. 2011 June; 52(6):1211-1221 describe LC/MS/MS methods for determining ceramide levels in cells.

In some embodiments, disclosed methods, such as contacting an oral squamous cell carcinoma with a ceramidase modulator, involve increasing ceramidase levels in the oral squamous cell carcinoma. Increased ceramidase protein levels resulting from disclosed methods can be determined according to known techniques, e.g., by Western blot, as described by Lucki et al., Molecular Endocrinology, 2012;26(2):228-243. In some embodiments, disclosed methods involve upregulating ceramidase genes in oral squamous cell carcinoma contacted by the ceramidase modulator.

Exemplary ceramidase genes, the expression of which can be measured to determine upregulation, include but are not limited to ASAH1, ACER2, and ACER3. A determination of ceramidase gene expression can be made according to methods available to one of skill in the art. For example, Yamada et al., J Cell Mol Med. 2023 May;27(9):1290-1295 and Kyriakou et al., Int. J. Mol. Sci. 2020, 21(5):1607 describes measuring the mRNA levels of ceramidases by RNA extraction and RT-PCR.

In some embodiments, disclosed methods involve contacting an oral cancer cell, such as an oral squamous cell carcinoma, a tongue squamous cell carcinoma, or a head and neck squamous cell carcinoma, with a ceramidase modulator comprising a sphingolipid derived from a bacterial cell. In some embodiments, the ceramide modulator includes a phosphorylated dihydroceramide derived from P. gingivalis or A. muciniphila. Methods for recovering and purifying lipids from Porphyromonas gingivalis are described, e.g., by Nichols et al., PLoS One, 2011. 6(2): p. e16771. Similar methods may also be used for recovering and purifying lipids from A. muciniphila. In some embodiments, the phosphorylated dihydroceramide is phosphoethanolamine dihydroceramide (PEDHC). In some embodiments, disclosed methods involve contacting an oral cancer cell with PEDHC. In some embodiments, the phosphorylated dihydroceramide is phosphoglycerol dihydroceramide (PGDHC). In some embodiments, disclosed methods involve contacting an oral cancer cell with PGDHC. In some embodiments, the sphingolipid is a15:0-15:0PE. In some embodiments, disclosed methods involve contacting an oral cancer cell with a15:0-15:0PE.

The lipids PGDHC and PEDHC lipids typically exist in high and low masses. The masses of lipids can be resolved using mass spectrometry, as described by Nichols et al., PLoS One, 2011. 6(2): p. e16771. P.gingivalis lipids can be extracted according to known methods, such as the methods described by Bligh & Dyer, J Biochem Physiol. 1959 August;37(8):911-7, fractionated via HPLC, and analyzed by GC/MS. As shown in FIG. 1A P. gingivalis phosphoglycerol dihydroceramides contain 3-OH isobranched (iso) C17:0 fatty acid in amide linkage to saturated dihydroxy long-chain bases of either 17, 18, or 19 carbons in length, and is substituted with isoC15:0 linked to the beta hydroxyl of 3-OH iso C17:0 (Nichols et al., J Lipid Res. 2004 December;45(12):2317-30). FIG. 1B shows that the dominant phosphatidyl-ethanolamine lipids are substituted with isoC15:0 with or without isoC13:0. See also Nichols et al., J Lipid Res. 2006 April;47(4):844-53 and Zahlten et al., J Dent Res. 2007 July;86(7):635-40. FIG. 1C shows A. muciniphila diacyl phosphatidylethanolamine with two C15:0 branched chains (a15:0-15:0PE).

In some embodiments, the oral cancer cell, such as the oral squamous cell carcinoma, tongue squamous cell carcinoma, gingival squamous cell carcinoma, or head and neck squamous cell carcinoma is derived from a biological sample. The biological sample can be any tissue, cell, fluid, or other biological material isolated from an organism, such as a mammalian organism. Preferably, the biological sample is derived from a human. In some embodiments, the sample comprises an oral tissue sample. In some embodiment, the sample is a biopsy. In another embodiment, the sample is a surgical biopsy, removed for example during an OSCC resection. In some embodiments, the biopsy is a punch biopsy, for example a 2 mm punch biopsy. In another embodiment, the test sample comprises histologically normal tumor resection margin tissue. In some embodiments, the sample comprises formalin fixed and/or paraffin embedded tissue, a frozen tissue or fresh tissue.

### Compositions

In some aspects, provided herein are compositions, such as pharmaceutical compositions including a ceramidase modulator. In some embodiments, the provided compositions can be used in connection with any of the methods described herein, e.g., methods of treating oral cancer cells, such as by contacting the oral cancer cells with a disclosed composition containing a ceramidase modulator. In some embodiments, disclosed pharmaceutical compositions are provided as unit dose form compositions for administration of a given dose or fraction thereof.

The pharmaceutical compositions generally include one or more optional pharmaceutically acceptable carrier or excipient. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. Compositions for in vivo administration are generally sterile. Sterility may be readily accomplished, e.g., by filtration through a sterile filtration membrane.

Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Disclosed compositions may include buffering agents. Suitable buffering agents include, e.g., citric acid, sodium citrate, phosphoric acid, potassium phosphate, and various other acids and salts. In some examples, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Disclosed compositions can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

Accordingly, there are a variety of suitable formulations. The pharmaceutical composition may also contain more than one active ingredients useful for the particular indication, disease, or condition being treated. In some embodiments, the pharmaceutical composition includes at least one additional therapeutic agent.

Disclosed compositions may be formulated for oral, intravenous, intraperitoneal, subcutaneous, pulmonary, transdermal, intramuscular, intranasal, buccal, sublingual, or suppository administration. In some embodiments, the disclosed compositions are administered parenterally. The term “parenteral,” as used herein, includes intravenous, intramuscular, subcutaneous, rectal, vaginal, and intraperitoneal administration.

Disclosed compositions may be provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Various additives which enhance the stability and sterility of the disclosed compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminium monostearate, and gelatin. Sustained-release preparations may also be prepared.

The compositions according to some embodiments of the present invention may be administered in an aqueous suspension, an oil emulsion, water in oil emulsion and water-in-oil-in-water emulsion, and in carriers including, but not limited to, creams, gels, liposomes (neutral, anionic or cationic), lipid nanospheres or microspheres, neutral, anionic or cationic polymeric nanoparticles or microparticles, site-specific emulsions, long-residence emulsions, sticky-emulsions, micro-emulsions, nano-emulsions, microspheres, nanospheres, nanoparticles and minipumps, and with various natural or synthetic polymers that allow for sustained release of the composition including anionic, neutral or cationic polysaccharides and anionic, neutral cationic polymers or copolymers, the minipumps or polymers being implanted in the vicinity of where composition delivery is required. Polymers and their use are described in, for example, Brem et al, Journal of Neurosurgery 1991;74:441-446. Furthermore, the active components of the compositions according to some embodiments of the present invention can be used with any carrier or a combination thereof. These include, but are not limited to, anti-oxidants, buffers, and bacteriostatic agents, and may include suspending agents and thickening agents.

For administration in a non-aqueous carrier, active components of the compositions according to some embodiments of the present invention may be emulsified with a mineral oil or with a neutral oil such as, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, soybean oil, canola oil, palm oil, olive oil and myglyol, wherein the number of fatty acid carbons is between 12 and 22 and wherein the fatty acids can be saturated or unsaturated. Optionally, charged lipid or phospholipid can be suspended in the neutral oil. More specifically, use can be made of phosphatidylserine, which targets receptors on macrophages. Use can be made of active components of the compositions according to embodiments of the present invention formulated in aqueous media or as emulsions using techniques known to those of ordinary skill in the art.

The compositions according to some embodiments of the present invention can comprise active agents described elsewhere in this document, and, optionally, other therapeutic and/or prophylactic ingredients. The carrier and other therapeutic ingredients must be acceptable in the sense of being compatible with the other ingredients of the composition and not deleterious to the recipient thereof.

The compositions according to some embodiments of the present invention are administered in an amount effective to induce a therapeutic response in an animal, including a human. The dosage of the composition administered will depend on the condition being treated, the particular formulation, and other clinical factors such as weight and condition of the recipient and route of administration.

In one embodiment, the amount of the composition administered corresponds from about 0.00001 mg/kg to about 100 mg/kg of an active component per dose. In another embodiment, the amount of the composition administered corresponds to about 0.0001 mg/kg to about 50 mg/kg of the active component per dose. In a further embodiment, the amount of the composition administered corresponds to about 0.001 mg/kg to about 10 mg/kg of the active component per dose. In another embodiment, the amount of the composition administered corresponds to about 0.01 mg/kg to about 5 mg/kg of the active component per dose. In a further embodiment, the amount of the composition administered corresponds to from about 0.1 mg/kg to about 1 mg/kg of the active component per dose.

Useful dosages of the compounds of the present invention can be determined by comparing their in vitro activity, and in vivo activity in animal models. Methods for the extrapolation of effective dosages in mice, and other animals, to humans are known in the art. For example, see U.S. Pat. No. 4,938,949.

### EXAMPLES

### Example 1: PGDHC Selectively Inhibits the Proliferative Activity of Human Gingival Squamous Carcinoma Cells (OECM1)

Applicant previously reported that PGDHC elevates inflammatory osteolysis and neuroinflammation in various experimental models. See, e.g., Duarte et al., J Cell Mol Med. 2022 May;26(10):2841-2851, Yamada et al., Front Immunol. 2020 Nov. 23:11:591571, and Ludwig et al., Int J Mol Sci. 2023 Jan. 3;24(1):877). PGDHC (0.1 μg/ml and 1 μg/ml) was tested in a concentration dependent manner to investigate its effects on the proliferation of oral squamous cell carcinoma.

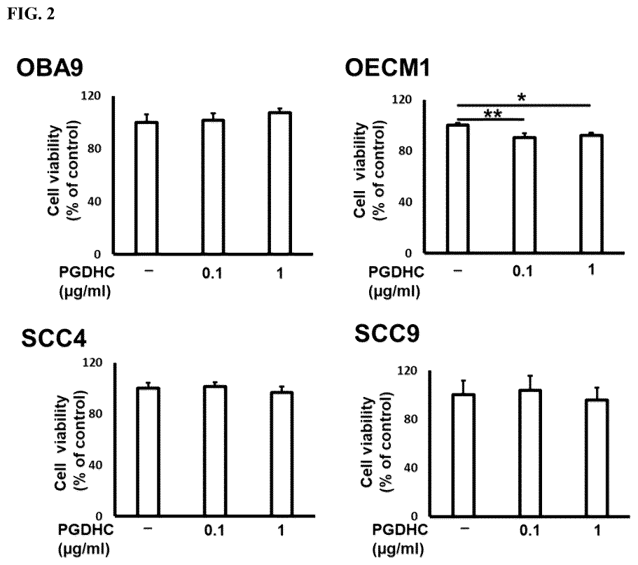

Various cell lines were exposed to PGDHC, including human gingival squamous carcinoma cells (OECM1), tongue squamous carcinoma cells (SCC4, SCC9), and healthy human gingival epithelial control cells (OBA9) in vitro. A WST-1 proliferation assay showed that each tested concentration of PGDHC significantly inhibited the viability of OECM1 cells (FIG. 2). In contrast, PGDHC did not have an observable effect on the proliferation of tongue squamous carcinoma cell lines SCC4 and SCC9, nor healthy control OBA9 cells (FIG. 2), indicating that PGDHC selectively inhibit OECM1 cells in vitro.

### Example 2: PGDHC Alters the Expression of Genes Implicated in Oral Carcinoma Cell Invasion and Inhibits Cell Transmigration

Invasion and migration of oral squamous cell carcinoma are implicated in the spread of cancer (Ellis, Cancers, 2021;13(11):2633). Cell invasion and migration involve modulation of the cell basement membrane and degradation of the extracellular matrix. To evaluate the potential of PGDHC to interfere with cell invasion and migration at a genetic level, the impact of PGDHC on the expression of genes involved in cell basement membrane and extracellular matrix degradation was determined. The effect of PGDHC on the migration behaviour of OECM1 cells was also assessed in vitro.

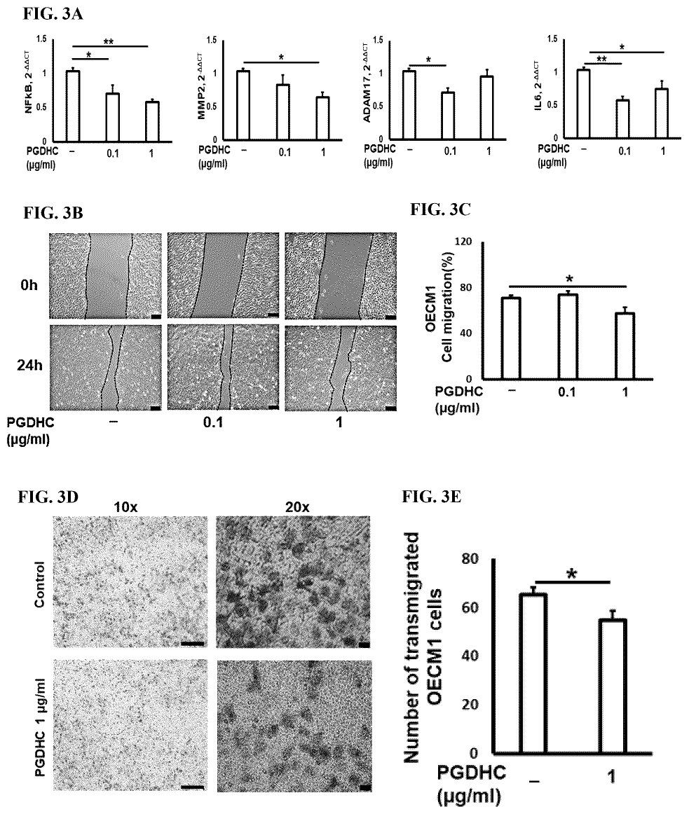

NF-kB, MMP-2, ADAM-17, and IL6 are implicated in the degradation of the expression of genes involved in the cell basement membrane and extracellular matrix of oral squamous cell carcinoma. Accordingly, the expression of NFkB, MMP2, ADAM17, and IL6 in OECM1 cells following exposure to 0.1 μg/ml PGDHC and 1 μg/ml PGDHC was determined. As shown in FIG. 3A, exposing OECM1 cells to 1 μg/ml PGDHC significantly inhibited expression patterns of NFkB, MMP2, and IL6 mRNA.

A scratch wound healing assay was performed according to known methods to assess the effects of PGDHC on the migration of human oral squamous cell carcinoma cells. The results showed that OECM1 migration was significantly inhibited by PGDHC at a concentration of 1 μg/ml compared to a sham control group (FIG. 3B and FIG. 3C). The scratch wound healing assay was validated with a transwell migration assay. The number of transmigrated OECM1 cells was significantly reduced in response to PGDHC compared to sham control cells in vitro (FIG. 3D and FIG. 3E).

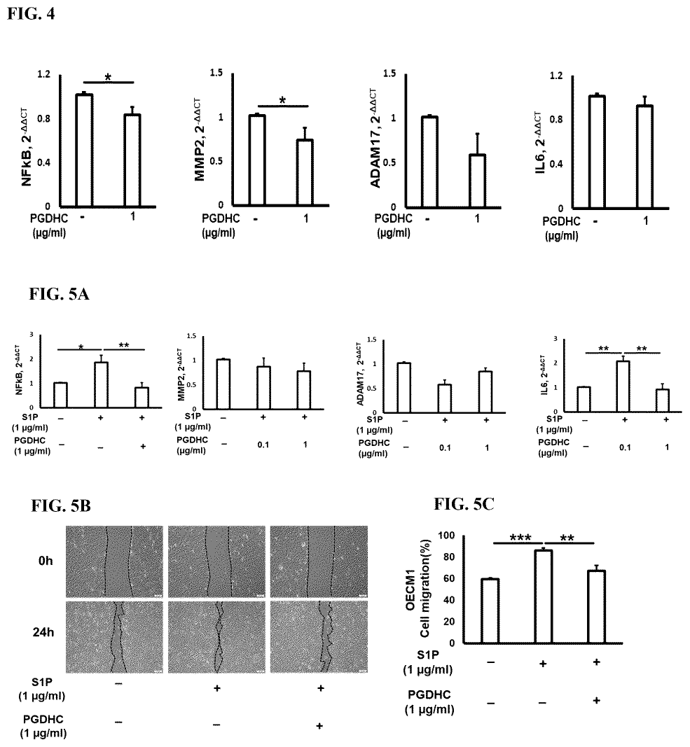

Expression of these genes was also assessed in human tongue squamous cell carcinoma SCC4 cells. Although PGDHC had no observable effect on the proliferation of tongue squamous cell carcinoma cells (see Example 1), PGDHC surprisingly altered gene expression in SCC4 cells. Relative to control cells, NFkB mRNA and MMP2 mRNA were significantly diminished in SCC4 tongue squamous cell carcinoma exposed to PGDHC (FIG. 4).

Together, these data indicate that PGDHC can interfere with the pathogenesis of oral squamous cell carcinoma by interfering with gene modulation of the cellular processes involved in cell invasion and migration.

### Example 3: PGDHC Interferes with Sphingosine-1-Phosphate-Mediated Cell Migration

Sphingosine-1-phosphate (S1P) sphingolipid plays an essential role in the survival, migration, and degradation of the basement membrane and extracellular matrix of oral squamous cell carcinoma (Yura et al., Cancers (Basel). 2020 August; 12(8): 2062). To determine whether PGDHC can inhibit such properties, the combined effects of PGDHC and S1P on OECM1 gene expression was tested in vitro.

PGDHC significantly inhibited the expression of NFkB and IL6 mRNA in OECM1 cells exposed to S1P (FIG. 5A). In contrast, a significant effect was not observed for MMP2 and ADAM17 mRNA levels in OECM1 cells exposed to PGDHC and S1P (FIG. 5A).

A wound healing migration assay was also conducted to determine the effects of S1P alone or in combination with PGDHC on cell migration. See Yamada et al., J Cell Mol Med. 2023 May; 27(9):1290-1295. The S1P-mediated wound-healing migration of OECM1 was dramatically inhibited by PGDHC compared to sham control cells (FIG. 5B and FIG. 5C). Together, these data indicate that PGDHC inhibits human oral squamous cell carcinoma OECM1 cell migration, even when exacerbated by S1P sphingolipid.

### Example 4: Determining the Effects of PGDHC on Cell Markers Involved in Metastasis

Various experimental evidence indicated that S1P contributes to the survival of cancer cells via the IL6/phosphorylated-STAT3 (p-STAT3) axis. See, e.g., Abaurrea et al., Int J Mol Sci. 2021 Aug. 3;22(15):8334, Nguyen et al., World J Gastroenterol. Aug. 14, 2014; 20(30): 10279-10287, Liang et al., Cancer Cell. 2013 Jan. 14;23(1):107-20, and Tamashiro et al., Cancer Cell Int. 2014 Aug. 29;14(1):76. Furthermore, the elevated levels of p-STAT3 also play a pivotal role in oral cancer cell metastasis via the Epithelial-Mesenchymal Transition (EMT) process which is mainly characterized by loss of the epithelial cell markers such as E-cadherin, N-cadherin, and gain of mesenchymal cell markers such as N-cadherin, and Vimentin. See, e.g., Han et al., Technol Cancer Res Treat. 2017 December;16(6):1209-121, and Sun et al., Mol Cancer. 2015 Dec. 21:14:213.

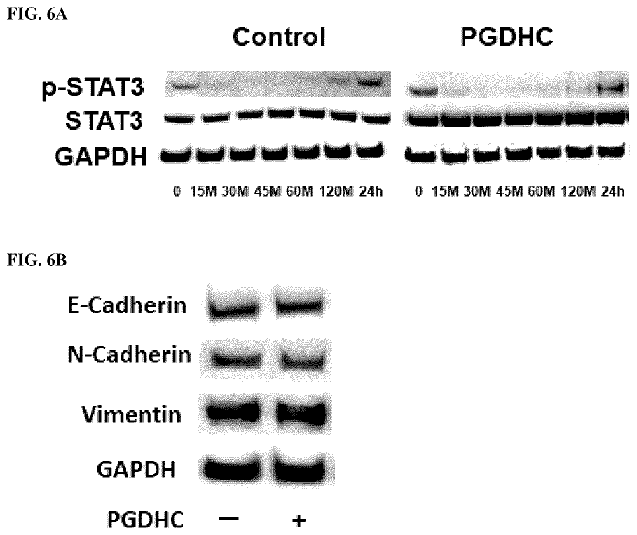

To determine the effect of PGDHC on cell markers involved in metastasis, the protein levels of p-STAT3, EMT markers, E-cadherin, N-cadherin, and Vimentin in OECM1 cells following in vitro exposure to PGDHC were determined by Western blot assay. Surprisingly, the time course study conducted between 15 minutes and 24 hours demonstrated no effect of PGDHC on the levels of p-STAT3 protein in OECM1 cells compared to the sham control (FIG. 6A). Furthermore, 24 h of treatment with PGDHC had little to no detectable effect on the protein levels of E-cadherin, N-cadherin, and Vimentin (FIG. 6B), indicating that PGDHC affects OECM1 cells survival and migration via an alternative downstream molecular mechanism.

### Example 5: PGDHC Promotes the Intracellular Accumulation of Antiproliferative Ceramides in Human OECM1 Oral Squamous Cell Carcinoma Cells

Applicant previously reported that a P. gingivalis-derived phosphoethanolamine dihydroceramide (PEDHC), with structural similarity to PGDHC, affects OECM1 cell catabolism via elevated accumulation of antiproliferative ceramides and inhibition of acid ceramidase (ASAH1) (Yamada et al., J Cell Mol Med. 2023 May;27(9):1290-1295). It was hypothesized that PGDHC may also inhibit oral squamous cell carcinoma proliferation and migration by affecting ASAH1 expression and accumulation of intracellular ceramides.

To test this hypothesis, it was first determined whether artificially elevated intracellular concentration of ceramide species affect the expression of acid/ASAH1, neutral/ASAH2, and alkaline1-3/ACER1-3 ceramidases in oral squamous cell carcinoma cells using C6-ceramide. Applicant previously reported that C6-ceramide serves as a positive control for PGDHC-mediated inflammation (Kanzaki et al., Biochim Biophys Acta Mol Cell Biol Lipids. 2017 May;1862(5):452-462) and promotes the intracellular accumulation of ceramide in cancer cells. See, e.g., Zhang et al., Front Plant Sci. 2020 Feb. 26:11:145, Zhai et al., Biochem Biophys Res Commun. 2015 December;468(1-2):274-80, Wilhelm et al., Cancers (Basel). 2021 Jan. 13;13(2):270, and Chapman et al., Biochem Pharmacol. 2010 Aug. 1;80(3):308-15.

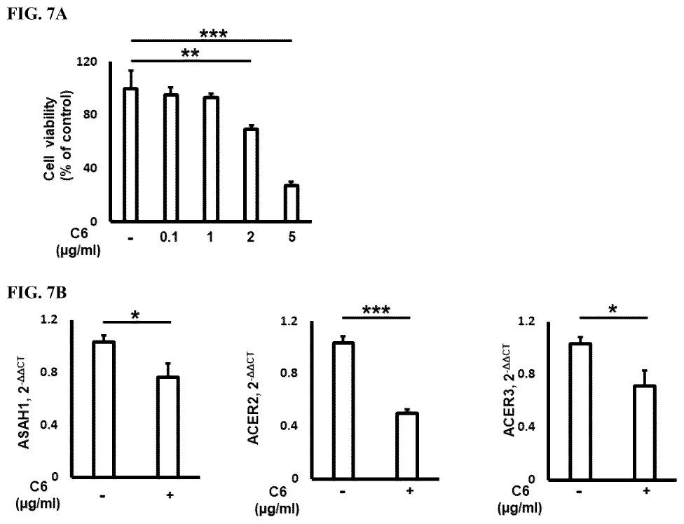

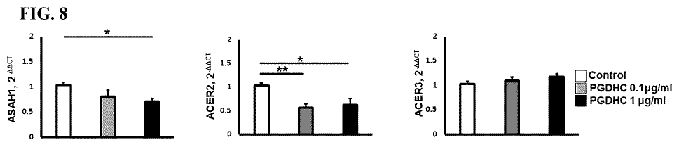

In a proliferation assay, C6 inhibited OECM1 viability in a concentration-dependent manner (FIG. 7A). Furthermore, C6-ceramide at a concentration of 1 μg/ml inhibited expression of ASAH1, ACER2, and ACER3 in OECM1 cells compared to the sham control group (FIG. 7B). Notably, neutral ceramidase expression/ASAH2 was not detected in OECM1 cells (Yamada et al., J Cell Mol Med. 2023 May;27(9):1290-1295; data not shown). Accordingly, these data indicate that C6-mediated accumulation of intracellular ceramides is critical for OECM1 cell proliferation and ceramidases (ASAH1/acid ceramidase and ACER2/alkaline ceramides-2) expression.

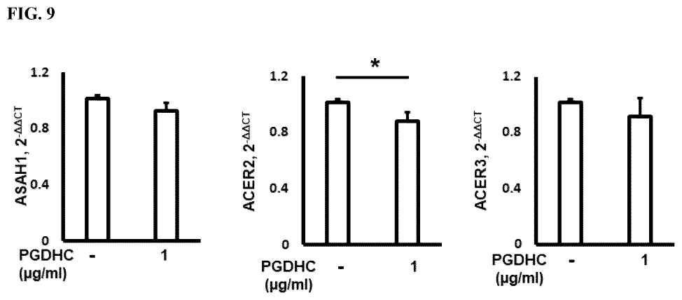

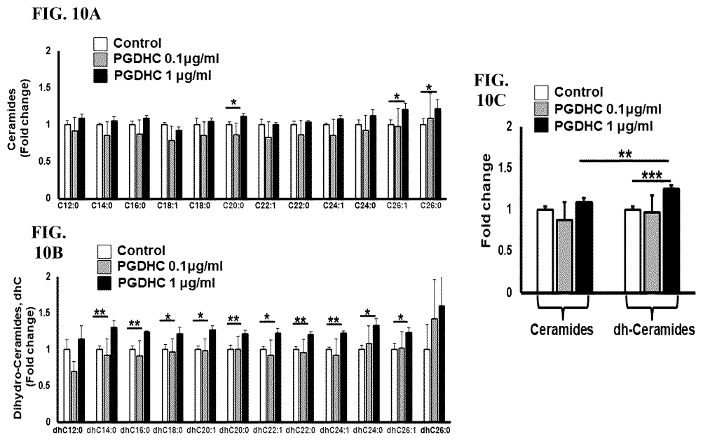

Next, the effect of PGDHC on ceramidase gene expression was determined. OECM1 and SCC4 cells were exposed to 0.1 or 1 μg/ml of PGDHC. The mRNA levels of ceramidases were then measured following PGDHC exposure. PGDHC inhibited the expression of ASAH1 and ACER2 mRNAs in OECM1 (FIG. 8). In addition, PGDHC downregulated the expression of ACER2 mRNA in SCC4 cells (FIG. 9). For context, it was previously demonstrated that diminished ceramidase expression, e.g., ASAH1 and ACER2, plays a significant role in the intracellular accumulation of ceramide and dihydroceramide sphingolipids in various cancer cells to induce cell death and enhance the antitumor responses. See, e.g., Vijayan et al., J Adv Res. 2023 Dec. 21:S2090-1232(23)00403-4, Doan et al., Oncotarget. 2017 Nov. 7;8(68):112662-112674., Lai et al., Sci Rep. 2021 May 27;11(1):11221, Liu et al., Cancer Sci. 2020 July;111(7):2259-2274., and Liu et al., Front Genet. 2023 Mar. 1:14:1148437.

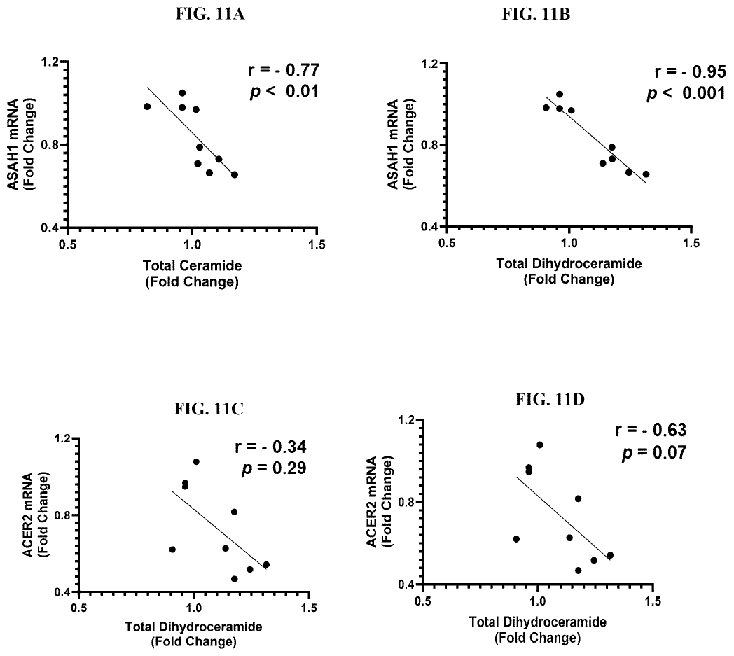

To assess intracellular ceramide levels, the concentration of ceramide and dihydroceramide in OECM1 cells exposed to PGDHC was measured using mass-spectrometry. Consistent with the preceding data, 1 μg/ml of PGDHC significantly elevated the intracellular concentration of ceramide C20:0, C26:0 and C26:1 (FIG. 10A) and dihydroceramide (dh) dhC14:0, dhC16:0, dhC18:0, dhC20:0, dhC20:1, dhC22:1, dhC22:0, dhC24:1, dhC24:0, dhC26:1 (FIG. 10B). In addition, the total intracellular concentration of dihydroceramides was significantly elevated in OECM1 cells compared to ceramides exposed to 1 μg/ml of PGDHC (FIG. 10C). No effect of 0.1 μg/ml of PGDHC on ceramide and dh-ceramide concentrations in OECM1 cells was observed. (FIG. 10C)

A potential correlation between the expression of ASAH1 and ACER2 ceramidases mRNA and the fold change of ceramide and dihydroceramide levels in OECM1 cells exposed to 1 μg/ml of PGDHC was investigated. (FIG. 11A-11D). There was little to no linear correlation between the levels of dihydroceramide and ACER2 mRNA expression, r=−0.63, p=0.07 (FIG. 11C) nor the levels of ceramide and ACER2 mRNA, r=−0.40, p=0.29 (FIG. 11D). In contrast, a significant negative linear correlation was determined between the levels of ceramide and ASAH1 mRNA, r=−0.07, p<0.01) (FIG. 11A), and the levels of dihydroceramide and ASAH1 mRNA expression, r=−0.94; p<0.001 (FIG. 11B).

Together, these results indicate that PGDHC promotes the accumulation of intracellular dihydroceramide via the downregulation of ASAH1/acid ceramidase in OECM1 oral squamous cell carcinoma cells in vitro.

### Example 6: Dihydroceramide C16:0 (dh-C16:0) Inhibits the Proliferation of OECM1 Cells and Modulates Gene Expression of Ceramidases

Growing evidence suggests that the accumulation of dihydroceramide is a potential therapeutic target for cancer cells. For example, ABTL0812 is an autophagy inducer that promotes cancer cell death by activation of cytotoxic autophagy selectively in tumor cells via inhibition of Akt/mTOR pathway. It is currently in clinical evaluation in a phase 2 trial for advanced squamous non-small cell lung carcinoma. See, e.g., Bosch-Barrera et al., Journal of Clinical Oncology. 2023; 41:16_suppl and Muñoz-Guardiola et al., Autophagy, 17:6, 1349-1366). Interestingly, ABTL0812 induces the accumulation of intracellular dihydroceramide accumulation, especially C16, C22, C24 and C24:1. Moreover, the application of photodynamic therapy (PDT), an anticancer therapy, lead to the accumulation of dihydroceramide species (C14-, C16-, C18-, C18:1-, C20:1-, and C22:1) in human head and neck squamous cell carcinoma UM-SCC-22A cells transfected with both control siRNA and dihydroceramide desaturase 1 (DES) (Breen et al., Anticancer Res. 2013 January;33(1):77-84).

However, the role of C16:0 and dh-C16:0 on oral squamous cell carcinoma is unclear. In one aspect, pro-survival molecule C16-ceramide has been shown to protect against ER-stress-induced apoptosis via selective regulation of the ATF6/CHOP axis and induce the growth of HNSCC tumor xenografts in vivo (Senkal et al., FASEB J. 2010 January;24(1):296-308). Additionally, data presented in the preceding example demonstrates that PGDHC inhibits expression of ASAH1 mRNA and promotes the accumulation of C16:0 dihydroceramide (dh-C16:0), but not C16:0 ceramide.

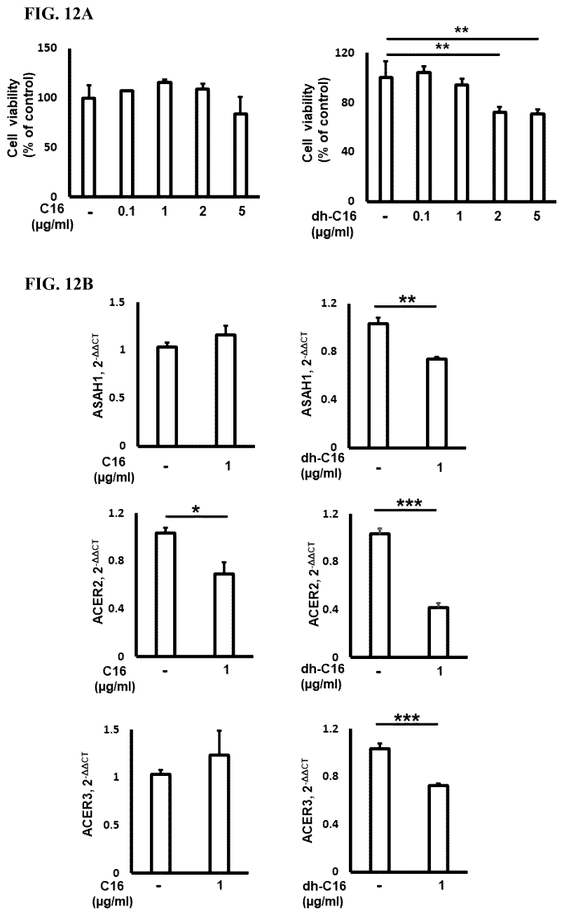

To elucidate the effects of C16:0 and dh-C16:0 on oral squamous cell carcinoma, the ability of exogenous C16:0 and dh-C16:0 to modulate proliferation and ceramidase expression in OECM1 cells was determined. Dh-C16:0, but not C16:0 ceramide, significantly inhibited OECM1 cells proliferation in a concentration-dependent manner (FIG. 12A). Importantly, FIG. 12B shows that dh-C16:0 also dramatically inhibits expression patterns of ASAH1 as well as ACER2, and ACER3 mRNA. In contrast, no effect of C16:0 on ASAH1 and ACER2 mRNA expressions.

### Example 7: Prevalence of Acid Ceramidase and Sphingosine-1-Phosphate (S1P) at the Lesion Site of Patients with Diagnosed Oral Squamous Cell Carcinoma (OSCC)

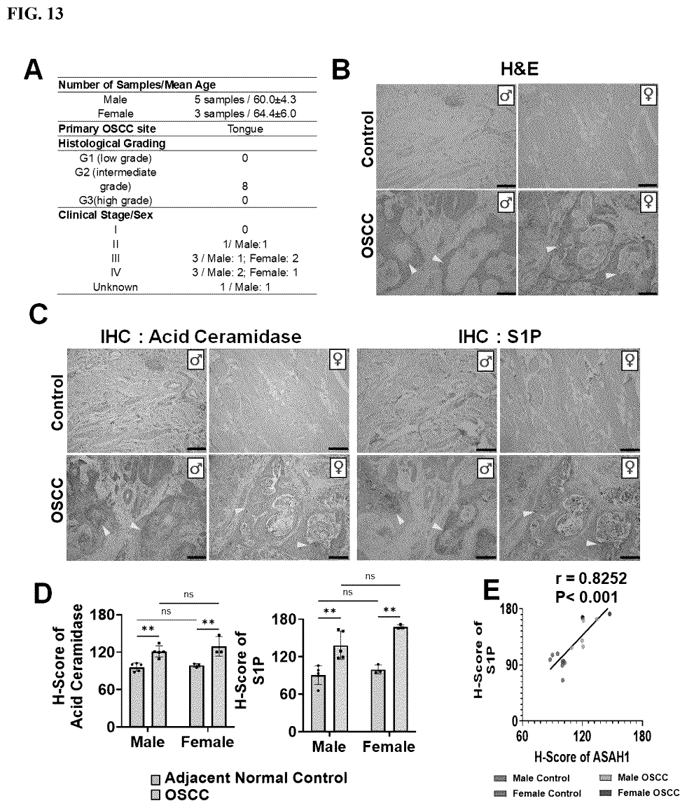

The prevalence of acid ceramidase and S1P was evaluated in samples obtained from patients with diagnosed oral squamous cell carcinoma (OSCC). As shown in FIG. 13A, samples were obtained from the tongues of 8 patients (5 male and 3 female). Six of the patients were at clinical stage III or IV, with 1 male patient at stage II and 1 male patient at an unknown clinical stage.

Hematoxylin & eosin (H&E) staining of tongue samples with OSCC lesions and matched adjacent normal control tissues were conducted and compared for each patient to confirm the presence of OSCC. (FIG. 13B). Once confirmed, immunohistochemical (IHC) staining for ceramidase and S1P of OSCC and healthy tongue samples for each patient was performed using an anti-acid ceramidase (Santa Cruz; dilution 1:200) and anti-S1P monoclonal antibodies (Abcam; dilution 1:200) (FIG. 13C). Quantification of the H-Score for both acid ceramidase and S1P in human OSCC, were confirmed higher levels in the OSCC samples compared to normal control tissues (FIG. 13D and FIG. 13E).

### Example 8: Evaluation of Acid Ceramidase Expression in OSCC Cells

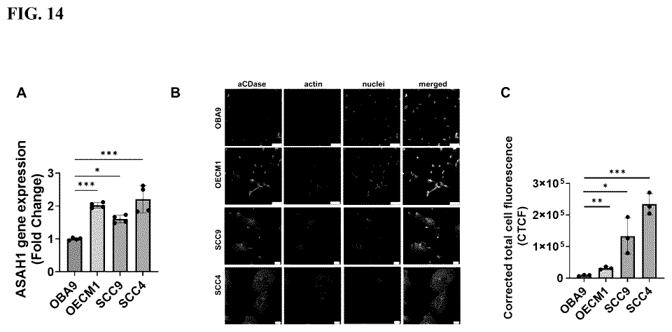

Expression of acid ceramidase in human oral squamous cell carcinoma (OSCC) cells compared to healthy epithelial cells was evaluated. First, expression patterns of ASAH1 mRNA in different OSCC, oral cavity (OECM1) and tongue (SCC9 and SCC4), and healthy epithelial OBA9, cell lines (OBA9) illustrated accelerated expression of acid ceramidase in OSCC cells compared to healthy epithelial cells in vitro (FIG. 14A). Levels of acid ceramidase were detected in different OSCC and healthy epithelial cells by confocal microscopy using acid ceramidase (FIG. 14B), and those levels were quantified as corrected total cell fluorescence based on the confocal images (FIG. 14C).

### Example 9: Evaluation of Acid Ceramidase Inhibitors

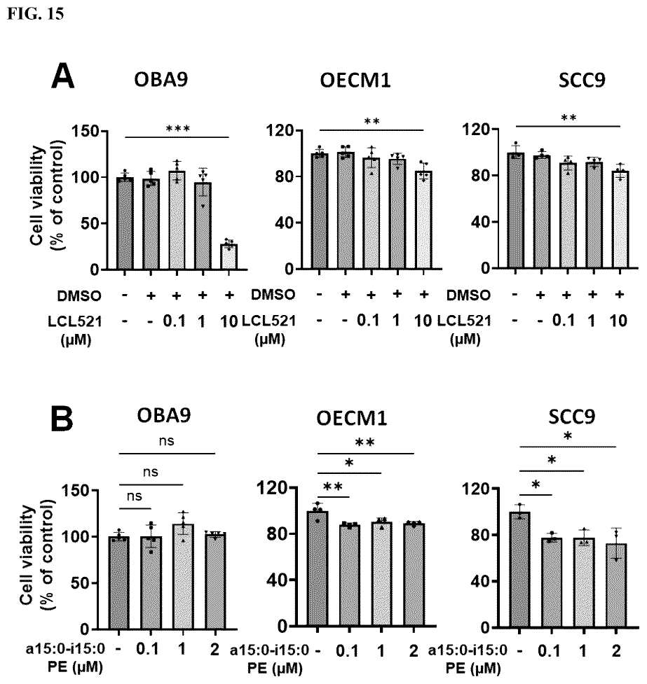

LCL521 is a dose-dependent acid ceramidase inhibitor. Comparison of the concentration-dependent effects of an acid ceramidase inhibitor, LCL521 (MedChemExpress®), and the bacterial-isolated a15:0-15:0PE phospholipid on the viability of healthy human epithelial, OBA9, and oral squamous cancer cell lines, OECM1 and SCC9, in vitro was evaluated. LCL521 significantly inhibited the viability of OBA9 by over 50%, as well as OECM1 and SCC9 by 20% at the concentration of 10 μM. (FIG. 15A). No significant effect of a 15:0-15:0PE on the healthy OBA9 cell viability was detected. In contrast, 15:0-15:0PE significantly inhibited the viability of OECM1 and SCC9 cells at concentrations lower than 1 μg/mL. (FIG. 15B).

### Example 10: Evaluation of Phosphoglycerol Dihydroceramide (PGDHC) Isolated from Porphyromonas gingivalis on Metabolic Pathways of OSCC

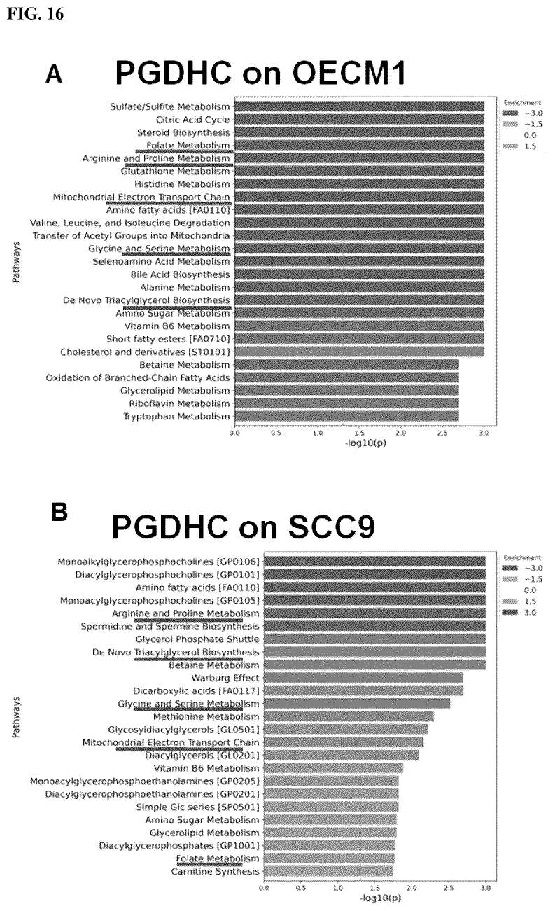

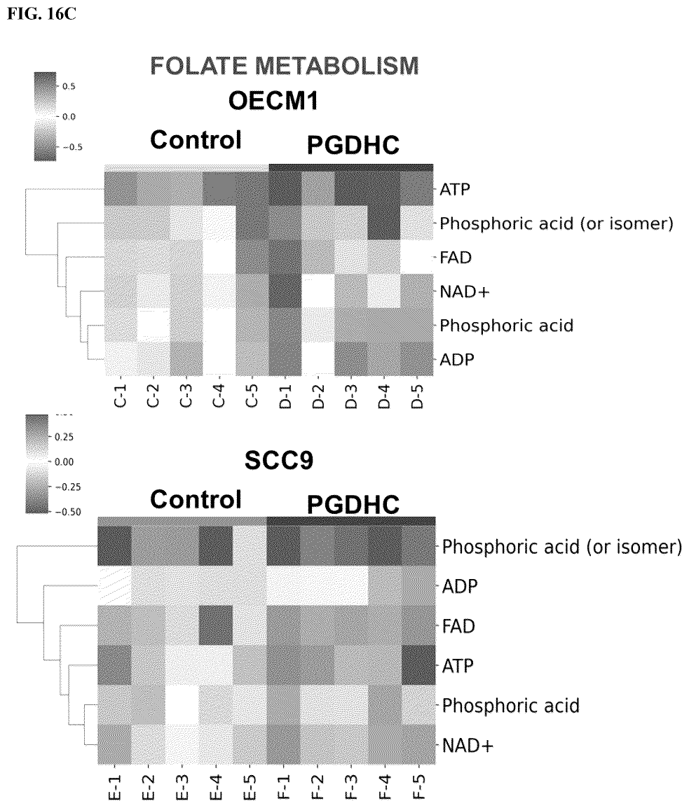

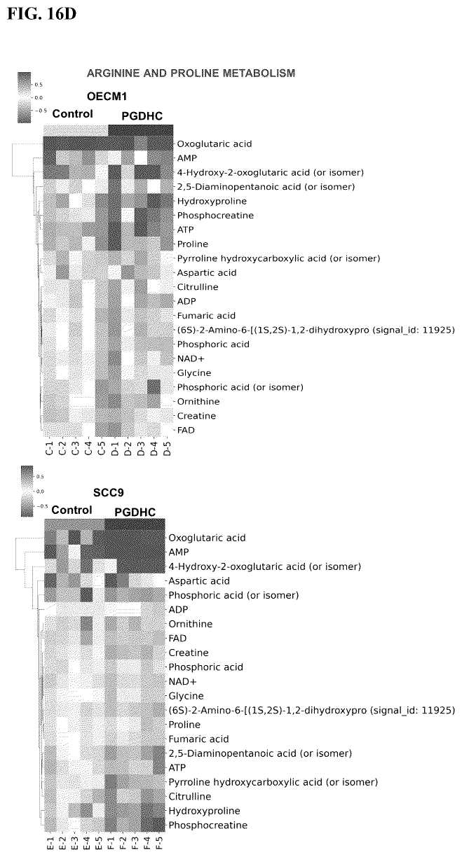

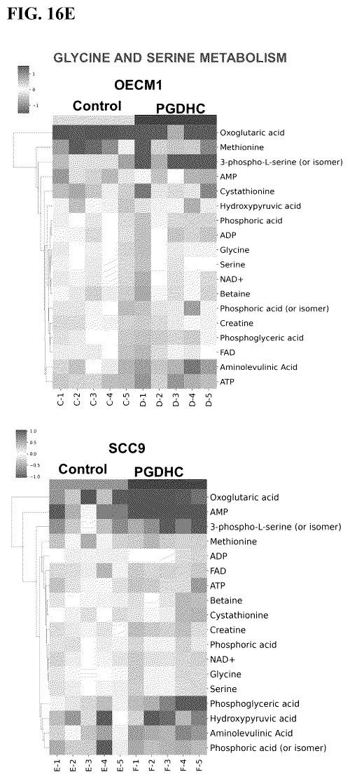

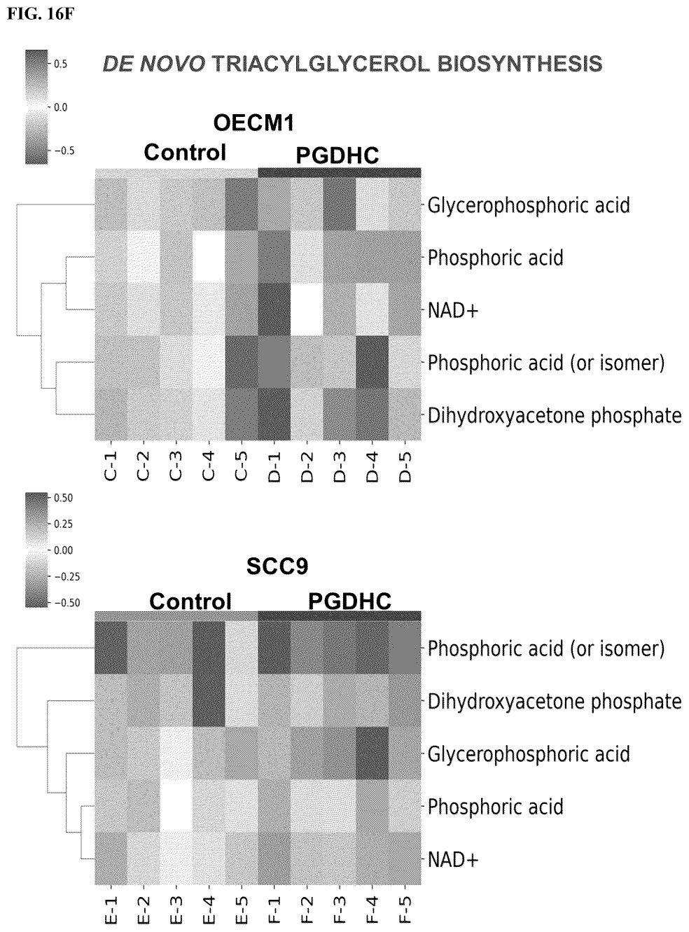

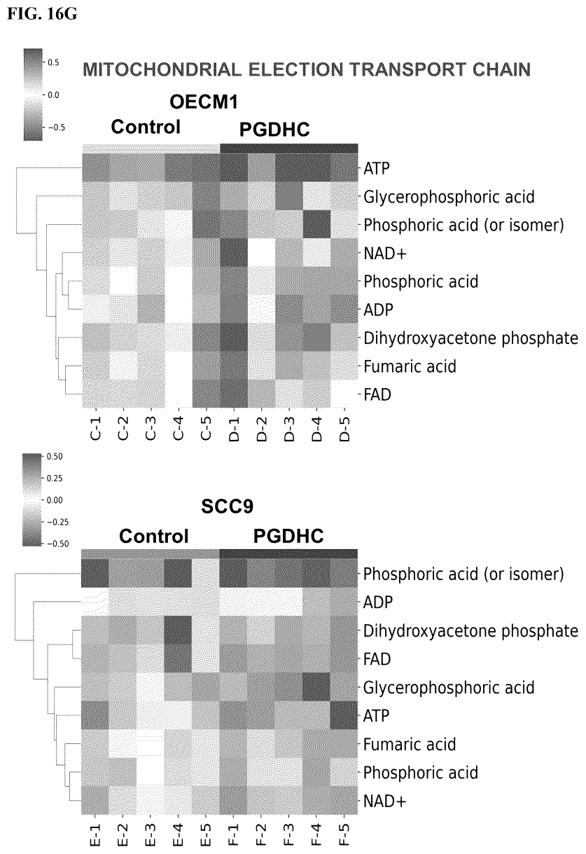

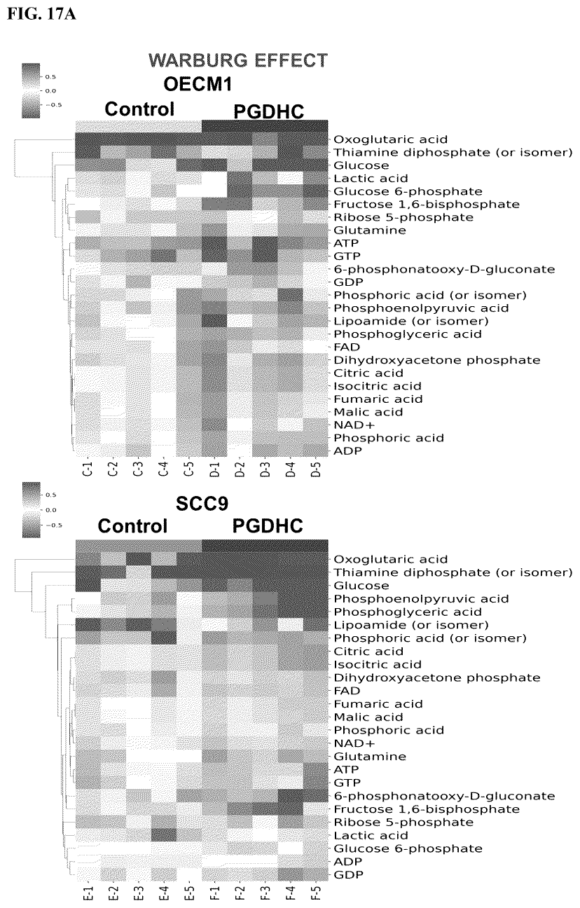

A pathway analysis of metabolites in OSCC (OECM1 and SCC9) and healthy OBA9 cells exposed to PGDHC (1 μg/ml) for 6 h in vitro was conducted to determine which, if any, metabolites were significantly altered (FIG. 16A). Based on this analysis, at least 5 different cell survival metabolomic pathways, underlined in red were diminished in OSCC (OECM1 and SCC9), but not in healthy epithelial OBA9 cells in response to PGDHC. The 5 pathways were folate metabolism, arginine and proline metabolism, glycine and serine metabolism, de novo triacylglycerol biosynthesis, and mitrochondrial electron transport chain.

The plots in FIGS. 16A and 16B display the enriched pathways identified in the analysis, with their corresponding significance levels represented on the x-axis. The significance threshold (p<0.05) is denoted by the dashed line. The size of the dots reflects the number of metabolites that showed statistical differences between the sample groups relative to the Control group (N=5/condition). Additionally, the color of the dots represents the enrichment, which indicates the extent to which the metabolites in the pathway were clustered amongst the compounds with the most positive fold-changes (positive enrichments), the most negative fold-changes (negative enrichments), or uniformly distributed across high and low fold-change compounds (enrichment values near 0.0). Heat map changes of the five cell survival metabolomic pathways demonstrate diminished accumulation of some metabolites required to folate metabolism (FIG. 16C), arginine and proline metabolism (FIG. 16D), glycine and serine metabolism (FIG. 16E), triacylglycerol biosynthesis de novo (FIG. 16F), mitochondrial electron transport (FIG. 16G), in OECM1 and SCC9 cells exposed to PGDHC. Polar and lipid metabolites were analyzed with hydrophilic interaction chromatography (HILIC/MS) and liquid chromatography (LC/MS), respectively. Mass spectrometry analysis was completed with a mass range of 67-1500 Da with 1 scan/sec and a mass resolution of 120,000 in both positive and negative ionization mode. The MS/MS mass-spectrometry data were acquired in a data-dependent iterative fashion with a 1.3 m/z isolation window. Based upon this metabolomic analysis, phosphoglycerol dihydroceramide (PGDHC) isolated from Porphyromonas gingivalis inhibits metabolomic pathways essential for cell survival in oral squamous cell carcinoma cells (OSCC).

### Example 11: Evaluation of Phosphoglycerol Dihydroceramide (PGDHC) Isolated from Porphyromonas gingivalis on Warburg Effect of OSCC

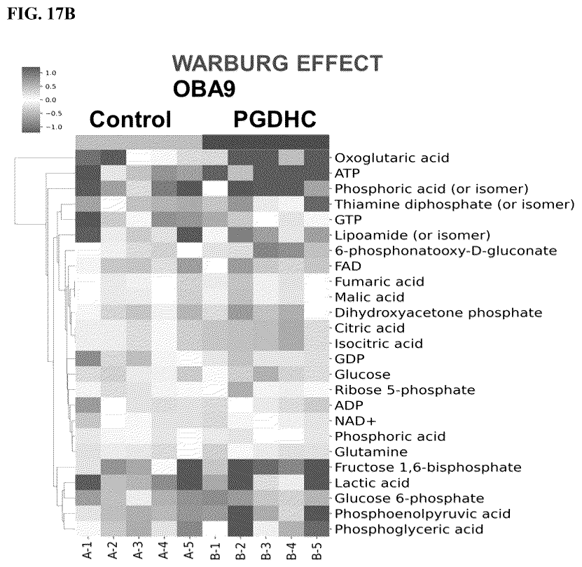

The Warburg effect is a metabolic phenomenon observed in many cancer cells, where the cells preferentially use glycolysis metabolism to produce energy instead of oxidative phosphorylation. The Warburg effect is a hallmark of rapid cancer cell growth. Therefore, we evaluated whether PGDHC altered Warburg effect in OSCM1 and SCC9 oral squamous cell carcinoma cells (FIG. 17A), but not healthy epithelial OBA9 cells (FIG. 17B), in vitro. Cells were stimulated with PGDHC (1 μg/ml) for 6 h and the untargeted metabolomic analysis was performed using LC/MS mass-spectrometry. Polar and lipid metabolites were analyzed with HILIC/MS and LC/MS, respectively. Mass spectrometry analysis was completed with a mass range of 67-1500 Da with 1 scan/sec and a mass resolution of 120,000 in both positive and negative ionization mode. MS/MS data were acquired in a data-dependent iterative fashion with a 1.3 m/z isolation window.

### Example 12: Evaluation of Alterations in Levels of ATP and Glucose in OSCC

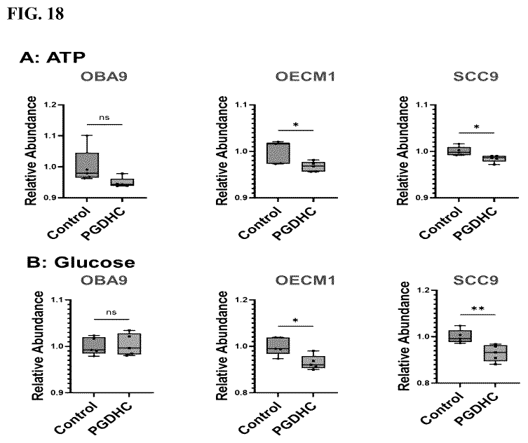

Based upon the impact of PGDHC on the Warburg effect in OSCC cells, we further analyzed the levels of cancer-survival metabolites adenosine triphosphate (ATP) and glucose in the OECM1, SCC9, and OBA9 cells described in Example 11. As shown in FIG. 18A and FIG. 18B, PGDHC altered the levels of both ATP and glucose in OECM1 and SCC9 oral squamous cell carcinoma cells, but not OBA9. The metabolites levels are presented as the relative abundance based on the data presented in FIG. 16 and FIG. 17 heat maps.

### Example 13: Evaluation of a15:0-15:0PE Phospholipid in OSCC

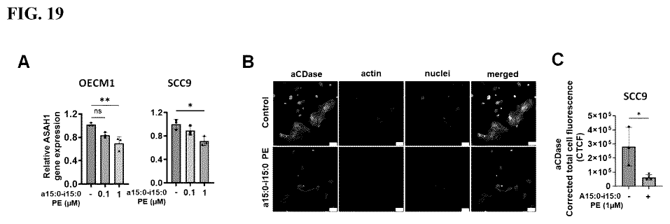

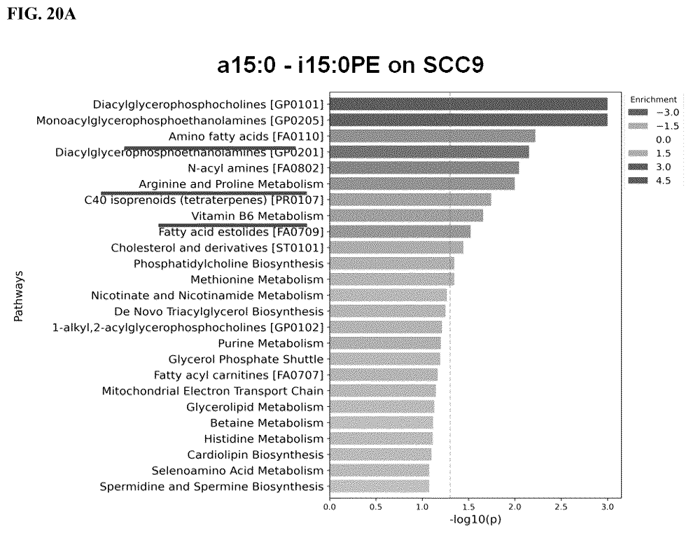

We evaluated the effect of a15:0-i15:0 phosphoethanolamine phospholipid (FIG. 1D), isolated from the gut bacteria Akkermansia muciniphila on OSCC cells. As shown in FIG. 19A, the expression patterns of ASAH1 mRNA in OECM1 and SCC9 exposed to different concentrations of a15:0-i15:0PE exhibited a dose-dependent decrease in expression. This decreased expression after exposure to a15:0-i15:0PE was also reflected in confocal microscopy images of SCC9 stained for acid ceramidase (green), actin (blue), nuclei (red) using anti-acid ceramidase Ab, phalloidin and Draq5, respectively (FIG. 19B) and the levels of acid ceramidase in the cells based on these confocal images (FIG. 19C). Based upon these results, a15:0-15:0PE inhibits acid ceramidase in SCC9 oral squamous cell carcinoma cells in vitro.

### Example 14: Evaluation of a15:0-i15:0PE Phospholipid in OSCC

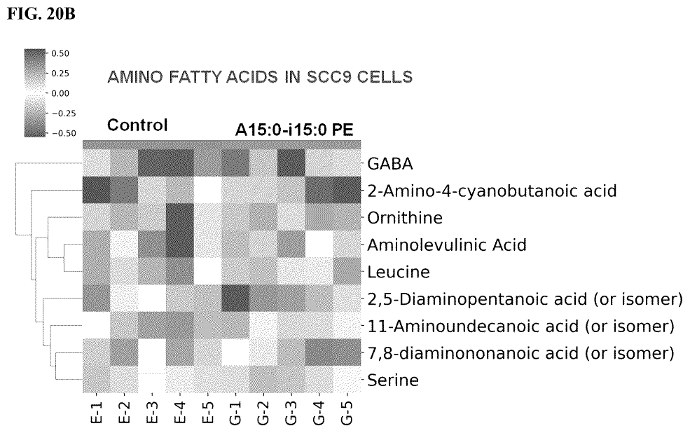

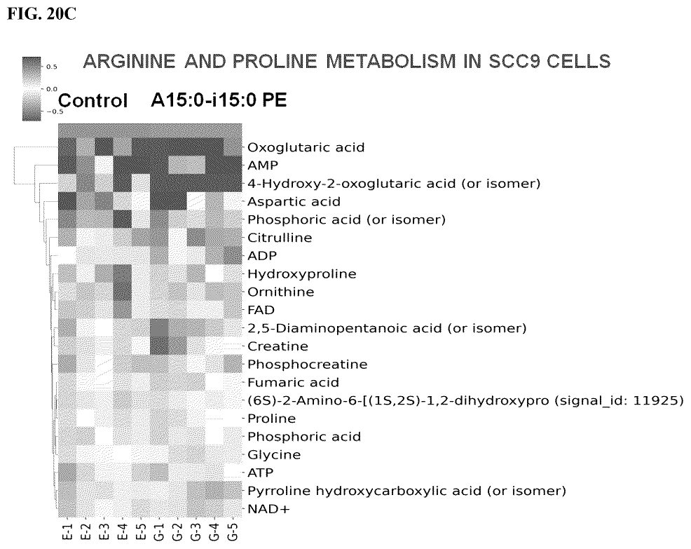

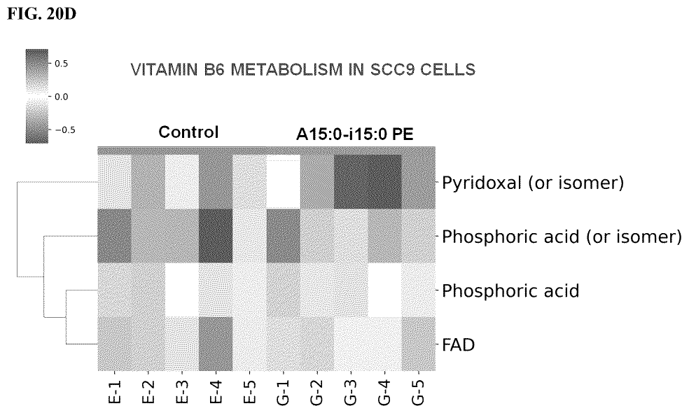

A pathway analysis was conducted to determine if a15:0-i15:0PE significantly altered metabolites OSCC cells. SCC9 were exposed to a15:0-i15:0PE (1 μg/ml) for 6 h in vitro. The plot displays of FIG. 20A reflect the enriched pathways identified in the analysis, with their corresponding significance levels represented on the x-axis. The significance threshold (p<0.05) is denoted by the grey dashed line. The size of the dots reflects the number of metabolites that showed statistical differences between the sample groups relative to the Control group (N=5/condition). Additionally, the color of the dots represents the enrichment, which indicates the extent to which the metabolites in the pathway were clustered amongst the compounds with the most positive fold-changes (positive enrichments), the most negative fold-changes (negative enrichments), or uniformly distributed across high and low fold-change compounds (enrichment values near 0.0).

Similarly, heat maps demonstrate a diminished accumulation in in human SCC9 tongue cancer cells exposed to a15:0-i15:0PE of some metabolites required for three metabolomic pathways: amino fatty acid metabolism (FIG. 20B), arginine and proline metabolism (FIG. 20C), and vitamin B6 metabolism (FIG. 20D).

The a15:0-i15:0PE phospholipid, isolated from the gut bacteria Akkermansia muciniphila, diminished three metabolomic pathways required for oral squamous cell carcinoma (OSCC) survival.

I claim:

1. A method for inhibiting viability of oral cancer cells comprising contacting the oral cancer cells with an effective amount of a ceramidase modulator, thereby inhibiting the viability of the oral cancer cells,
wherein the ceramidase modulator comprises a phosphorylated dihydroceramide.

2. The method of claim 1 wherein the phosphorylated dihydroceramide comprises phosphoethanolamine dihydroceramide (PEDHC), phosphoglycerol dihydroceramide (PGDHC), a15:0-i15:0 phosphoethanolamine, or an analog thereof.

3. The method of claim 1, wherein the method comprises increasing the concentration of a ceramide and/or a dihydroceramide in the oral cancer cells.

4. The method of claim 3, wherein the ceramide is selected from the group consisting of C20:0, C26:0, and C26:1; and
the dihydroceramide is selected from the group consisting of dhC14:0, dhC16:0, dhC18:0, dhC20:0, dhC20:1, dhC22:1, dhC22:0, dhC24:1, dhC24:0, and dhC26:1.

5. The method of claim 1, wherein the method comprises inhibiting the gene expression of a ceramidase in the oral cancer cells.

6. The method of claim 5, wherein the ceramidase is selected from ASAH1 and ACER2.

7. The method of claim 1, wherein the oral cancer cells comprise oral squamous cell carcinoma, tongue squamous cell carcinoma, head and neck squamous carcinoma, or a combination thereof.

8. A pharmaceutical composition comprising a ceramidase modulator and a pharmaceutically acceptable excipient,
wherein the ceramidase modulator comprises a phosphorylated dihydroceramide.

9. The pharmaceutical composition of claim 8 wherein the phosphorylated dihydroceramide comprises phosphoethanolamine dihydroceramide (PEDHC), phosphoglycerol dihydroceramide (PGDHC), a15:0-i15:0 phosphoethanolamine, or an analog thereof.

10. The composition of claim 8, wherein the phosphorylated dihydroceramide is purified from a bacterial cell.

11. The composition of claim 9, wherein the phosphorylated dihydroceramide is purified from Porphyromonas gingivalis.

12. The composition of claim 9, wherein the phosphorylated dihydroceramide is purified from Akkermansia muciniphila.

13. The composition of claim 8, wherein the composition comprises phosphoethanolamine dihydroceramide (PEDHC), phosphoglycerol dihydroceramide (PGDHC), a15:0-i15:0 phosphoethanolamine, an analog thereof, or a combination thereof.

14. A method of treating oral cancer comprising contacting the oral cancer cells of a subject with an effective amount of a ceramidase modulator,
wherein the ceramidase modulator comprises a phosphorylated dihydroceramide.

15. The method of claim 14 wherein the phosphorylated dihydroceramide comprises phosphoethanolamine dihydroceramide (PEDHC), phosphoglycerol dihydroceramide (PGDHC), a15:0-i15:0 phosphoethanolamine, or an analog thereof.

16. The method of claim 14, wherein the subject has mouth cancer, lip cancer, tongue cancer, head and neck cancer, or a combination thereof.

17. The method of claim 14, wherein the subject is a mammal, and wherein the mammal is a human.
